(19) United States
(12) Patent Application Publication
(10) Pub. No.: US 2022/0084670 A1
(43) Pub. Date: Mar. 17, 2022

(54) DIGITAL HEALTHCARE SYSTEMS AND METHODS FOR PATIENT CARE

(71) Applicants: Alyssa Jones, Fishers, IN (US); Bruce Jones, Valparaiso, IN (US)

(72) Inventors: Alyssa Jones, Fishers, IN (US); Bruce Jones, Valparaiso, IN (US)

(21) Appl. No.: 17/019,736

(22) Filed: Sep. 14, 2020

(51) Int. Cl. G16H 40/67; G06F 3/0481; G06F 3/03; H04L 29/08; G16H 70/20; G16H 10/60

(52) U.S. Cl. CPC G16H 40/67; G06F 3/04817; G16H 10/60; H04L 67/12; G16H 70/20; G06F 3/03

(57) ABSTRACT

A digital healthcare system for patient care includes at least hardware, software, data storage, and a network for interconnecting components of the system. The system includes a cross-platform software for controlling an interactive display device for a patient being treated. The interactive display device, having a default home screen, facilitates access to a plurality of electronic cognitive aids. The system also includes a software application for receiving input from a plurality of different designated users (e.g., at least one healthcare professional, caregiver, family member, or power of attorney). The default home screen is customizable based on the collaborated care or user preferences using the software application.

This specification relates to the field of software. More specifically, the present technology is in the technical field of software-based digital healthcare systems.

Cognitive impairments may range from mild to severe, with some degree of cognitive impairments being a common part of aging, some relating to an acute medical condition, and some relating to diseases that cause progressive damage to the brain. Cognitive impairments may include deficits with attention, memory, executive functioning, processing of information, and/or language.

Oftentimes, people with cognitive impairments may benefit from the use of external memory aids. External memory aids include, for example, memory books, calendars, photographs, and orientation signs, and are frequently used to help people with cognitive impairments orient to their environment and remember important information.

The present novel technology, which may be implemented to address memory impairments, targets various existing needs in patient care.

### SUMMARY

This specification relates to the field of software. More specifically, the present technology is in the technical field of software-based digital healthcare systems.

The details of one or more embodiments of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 depicts an environment in which a digital healthcare system may operate, according to an exemplary embodiment of the present disclosure.

FIG. 2 is an exemplary screen, or home screen, of an interactive display device of the digital healthcare system that may be viewed by a user, such as a patient, according to a first configuration.

FIG. 3 is an alternative exemplary screen, or home screen, having a different configuration relative to FIG. 2.

FIG. 4 is another alternative screen, or home screen, having a different configuration relative to both FIGS. 2 and 3.

FIG. 5 is an exemplary screen that may be displayed in response to the patient tapping a button on the home screen.

FIG. 6 is another exemplary screen that may be displayed in response to the patient tapping a button on a home screen.

FIG. 7 is an exemplary screen for a user to input information to be displayed for the patient via a user device.

Like reference numbers and designations in the various drawings indicate like element.

### DETAILED DESCRIPTION

Before the present methods, implementations, and systems are disclosed and described, it is to be understood that this invention is not limited to specific synthetic methods, specific components, implementation, or to particular compositions, and as such may, of course, vary. It is also to be understood that the terminology used herein is for the purpose of describing particular implementations only and is not intended to be limiting.

As used in the specification and the claims, the singular forms “a,” “an” and “the” include plural referents unless the context clearly dictates otherwise. Ranges may be expressed in ways including from “about” one particular value, and/or to “about” another particular value. When such a range is expressed, another implementation may include from the one particular value and/or to the other particular value. Similarly, when values are expressed as approximations, for example by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. Similarly, “typical” or “typically” means that the subsequently described event or circumstance often though may not occur, and that the description includes instances where said event or circumstance occurs and instances where it does not. Additionally, “generates,” “populates,” “generating,” and “populating” mean that rental property management system or module may produce some event or cause some event element to be produced. For example, a webpage may receive data to display in whole or in part to display a valuation estimate to an end digital, the webpage may pull such data from a source other than system (e.g., other servers, intermediaries, etc.), or system may entirely provide the valuation estimate to be produced on the webpage.

“Click,” “touch,” and “tap” are terms that typically may be used interchangeably. For example, on a mobile device, users typically touch or tap the screen to select items or press buttons. On a laptop, smartphone, tablet, and/or desktop users typically click with a mouse but may in some implementations use a touch screen or the like interface.

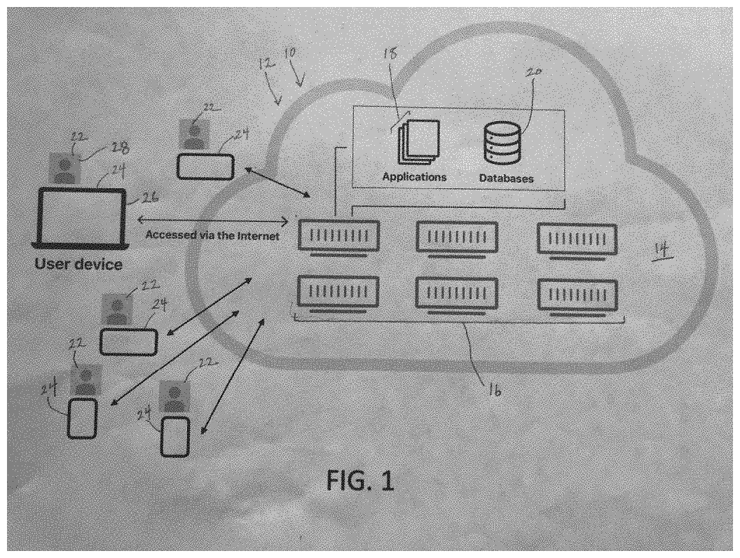

FIG. 1 is a block diagram 10 of an exemplary environment in which the digital healthcare system 12 of the present disclosure may operate. The exemplary environment, which may or may not utilize “the cloud 14,” may include a plurality of servers 16, along with software 18 and databases 20 capable of running on the servers 16. Software and/or applications 18 implementing the digital healthcare system 12 of the present disclosure may be hosted on one or more of the servers 16. According to some embodiments, the software 18 may be a cross-platform software, which may be implemented on multiple computing platforms.

The digital healthcare system 12 may be implemented using a variety of different digital environments and should not be limited to the exemplary embodiment that is provided herein. For example, the digital healthcare system 12 may be a web based or cloud based application, depending on the particular implementation that is desired. Further, major components of the digital environment may include websites, cloud servers, search engines, social media outlets, mobile apps, audio and video, and other web-based resources, to name a few.

Various users 22, using any one of a variety of different user devices 24, may access the digital healthcare system 12 as described herein, such as, for example, via the Internet. Exemplary user devices 24 may include desktop computers, mobile phones, tablets, smart TVs, etc. With specific reference to the digital healthcare system 12, one of the user devices 24 may include an interactive display device 26 and the types of users may generally include, for example, patients, healthcare professionals, caregivers, family members and/or those with power of attorney. Additional and/or alternative users may also use the digital healthcare system 12 with respect to any of a variety of different functionalities and/or services offered by the digital healthcare system 12.

The one or more databases 20 may be used to store a variety of information needed for operating the digital healthcare system 12, including, for example, user accounts and/or profiles, settings, preferences, and various other information that may or may not be unique to the digital healthcare system 12 and to implementing its functionality. Types of information that may be stored may include, for example, images, text and media files.

The digital healthcare system 12 may be a patient-facing system that is designed to provide a wide range of computer or Internet-based services that support patient interactions with various users of the digital healthcare system 12, such as one or more of the users mentioned above. For instance, and according to the exemplary embodiment, the digital healthcare system 12 may be configured to provide a unique combination of electronic cognitive aids, also referred to as external memory aids, for people, such as the patient 28 with memory impairments, at bedside or at home, or at another location where the digital healthcare system 12 would prove beneficial. The electronic cognitive aids may include memory aids, communication aids and/or audio and visual reminders. According to the present disclosure, the electronic cognitive aids may be integrated into the digital healthcare system 12, as described below.

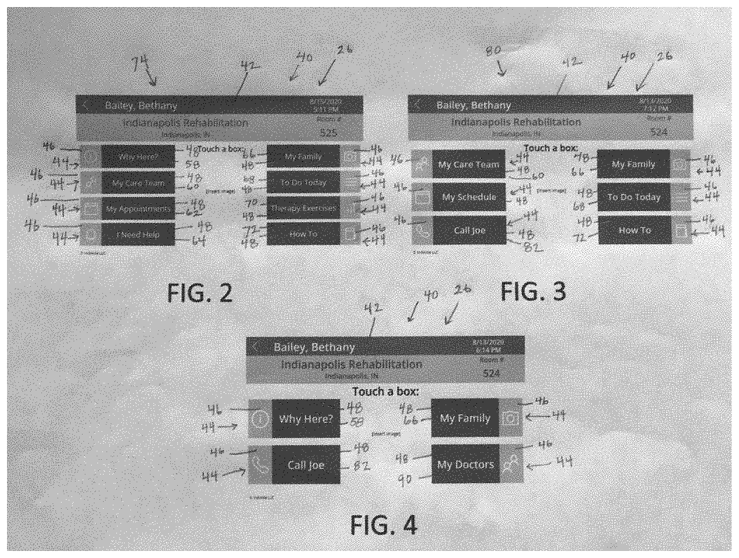

FIG. 2 is an exemplary screen 40, or home screen, of an interactive display device 26, which may also be referred to as a user device 24, of the digital healthcare system 12, that may be displayed for a user 22. Although a number of different users 22 may benefit from the digital healthcare system 12 disclosed herein, one of the users 22 is often referenced as a patient 28 throughout. The screen 40, which may be a touchscreen, may include a title bar 42, including, for example, date/time and other information that may be personal to the patient 28. For example, the title bar 42 may display the name of the patient 28, such as a proper or common name for a location and additional identifying information, such as a room number. There may also be an indication of where the patient 28 is located, such as at home or in a facility, displayed in the title bar 42. It should be appreciated that the information provided in the title bar 42, and contents thereof, may be modified, depending on the particular patient 28.

The screen 40 may also include one or more buttons 44, which may include icons 46 and/or text 48, for navigating, or directing, the patient 28 to content provided by one of a plurality of dedicated users 22, such as, for example, healthcare professionals 50, caregivers 52, family members 54 and someone having power of attorney 56. According to the exemplary embodiment of the present disclosure, the dedicated users 50, 52, 54 and 56 may contribute content personalized for the patient 28 that is accessed and viewed by the patient 28. Content may relate to the relationship the dedicated user 50, 52, 54 and 56 has with the patient 28.

The buttons 44 may include icons 46 that correspond to the accompanied text 48, For instance, and according to the present embodiment, the screen 40 may include the following text 48, paired with a matching icon 46: “Why Here?,” “My Care Team,” “My Appointments,” “I Need Help,” “My Family,” “To Do Today,” “Therapy Exercises,” and “How To,” Although particular embodiments are shown and described throughout the specification, this disclosure is not intended to be limited to these embodiments.

The “Why Here?” button 58, which includes an icon 46 and text 48, if selected by the patient 28, may direct the patient 28 to personalized content. For example, the content may include the location of the patient 28, such as, for example, at home, at a healthcare facility or at a hospital, and a description or reason for the current location of the patient 28.

The “My Care Team” button 60, which includes an icon 46 and text 48, if selected by the patient 28, may direct the patient 28 to content regarding a current care team for the patient 28. The patient 28 may have a care team responsible for monitoring and managing the health and welfare of the patient 28, particularly if the patient 28 is in a hospital, health care facility, or in a home health care arrangement.

The “My Appointments” button 62, which includes an icon 46 and text 48, if selected by the patient 28, may direct the patient 28 to different content, such as, for example, scheduled appointments for the day, the week, etc. Information regarding who the appointment is with, the location of the appointment, and date/time of the appointment may be provided. Additional or alternative information may be provided, depending on the specific needs of the patient 28 and depending on the type of appointment.

The “I Need Help,” button 64, which could alternatively read “Speak” or “Talk,” includes an icon 46 and text 48, if selected by the patient 28, may direct the patient 28 to different content and/or may automatically route a message to a predetermined recipient, notifying one or more users 22 recipient regarding the need of the patient 28 for assistance. Further, when the button 64 is selected, the patient 28 may be routed to a different screen 40 having a plurality of buttons that correspond to predetermined messages. The patient 26 may select any of the predetermined messages to submit the selected predetermined message to the predetermined recipient using speech generation. Further, when the speech generation button is selected, a voice output of the message may be produced for the patient 28.

The “My Family” button 66, which includes an icon 46 and text 48, if selected by the patient 28, may direct the patient 28 to different content, such as, for example, family content including photos and/or other content that might help aid memory with specific regard to family, friends, and important life events.

The “To Do Today” button 68, which includes an icon 46 and text 48, if selected by the patient 28, may direct the patient 28 to different content, such as notes or lists, which may assist the patient 28 in remembering and organizing tasks to be completed.

The “Therapy Exercises” button 70, which includes an icon 46 and text 48, if selected by the patient 28, may direct the patient 28 to different content, such as an identification of exercises and other health related activities the patient 28 is tasked to complete.

The “How To” button 72, which includes an icon 46 and text 48, if selected by the patient 28, may also direct the patient 28 to different content. For example, the content may include instructions for tasks regularly completed by the patient 28. In addition, this content may include a tutorial of how to use the software itself.

The screen 40 of FIG. 2 may be a default home screen 74 provided with the interactive display device 26 of the digital healthcare system 12. A default home screen 74 may be provided, with options to customize content, as needed.

For example, FIG. 3 illustrates an alternative home screen 80 of the interactive display device 26 of the digital healthcare system 12, that may be displayed for a patient 28. The screen 40, which may also be referenced as alternative home screen 80, may include a title bar 42 similar to that of FIG. 2, including content that is personal and/or informational.

The screen 40, or alternative home screen, of FIG. 3 may include the following buttons 44: “My Care Team,” “My Schedule,” “Call Joe,” “My Family,” “To Do Today,” and “How To.” Similar buttons 44 may navigate to similar content, with respect to FIG. 2, personalized for the particular patient 28. It should be appreciated that the screen 40 may be highly customized, including a customization of the buttons 44, the arrangement of the buttons 44, the title bar 42, etc.

The “Call Joe?” button 82, which includes an icon 46 and text 48, if selected by the patient 28, may initiate a video call with a designated recipient. Alternatively, the patient 28 may be directed to another screen 40 where the patient 28 may be presented with a number of different designated recipients the patient may select to call. The “How To” button 44, which includes an icon 46 and text 48, if selected by the patient 28, may direct the patient 28 to different content, which may include instructions for completing common tasks.

FIG. 4 illustrates another alternative home screen 80 of the interactive display device 26 of the digital healthcare system 12, that may be displayed for the patient 28. The screen 40, also reference as an alternative home screen 80, may include a title bar 42 similar to the title bars 42 of FIGS. 2 and 3, including content that is personal and/or informational.

The alternative home screen, or screen, 80 of FIG. 4 may include the following buttons: “Why Here” button 58, “My Family” button 66, “Call Joe” button 82, and “My Doctors” button 90. Similar buttons 44 may navigate to similar content, with respect to FIGS. 2 and 3, that is personalized for the particular patient 28. It should be appreciated that the alternative home screen 80, or screen 40, may be highly customized, including a customization of the buttons 44, icons 46, and text 48, including arrangement and associated content. The “My Doctors” button 90 which includes an icon 46 and text 48, if selected by the patient 28, may direct the patient 28 to different content, which may include an identification and contact information for doctors of the patient 28, along with options for sending or receiving correspondence.

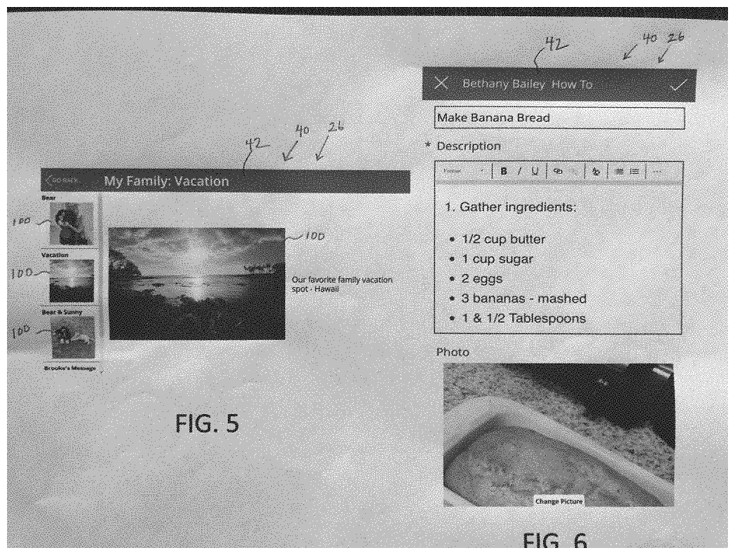

FIG. 5 is another alternative home screen 80, or screen 40, that may be displayed in response to the patient 28 tapping a button 44 on the home screen, or screen, 40. In particular, a patient 28, or other user 22, may tap the “My Family” button 66 and be directed to the screen 40 shown in FIG. 5. A title bar, similar to title bar 42, may be provided that includes useful information for the patient 28, including a button 44 to return back to the home screen 40. As shown, the screen 40 of FIG. 5 may display various family content. For example, photographs 100 of various family members 54, friends and memories may be added. This is merely provided as an example. Additional or alternative content may also be displayed. Further, a family member 54 may input this information into the digital healthcare system 12.

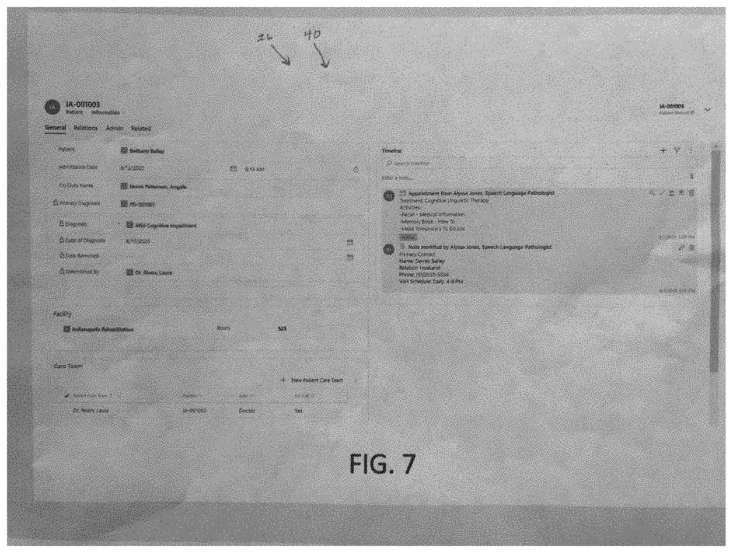

FIG. 6 is another exemplary screen 40 that may be displayed in response to the patient 28 tapping a button 44 on the home screen 40. When tapping the “How To” button 72, for example, the patient 28 may be directed to content provided by a user 22, such as a family member 54. As described above, the content may include instructions, or directions, for tasks regularly completed by the patient 28, such as, for example, following a recipe. In addition, this content may include a tutorial of how to use the software itself. For example, the tutorial may include instructions for using the interactive display device 26.

FIG. 7 is a screen 40 for a user 22 to input information to be displayed for the patient 28 on the interactive display device 26. According to the present disclosure, the contributed content may be accessed by the patient 28 using one or more buttons provided on the home screen 40.

One of a plurality of different users 22 may input data specific to the patient 28 into the digital healthcare system 12, such as by using the screen 40 of FIG. 7. The users 22 may include, for example, healthcare professionals 50, caregivers 52, family members 54 and/or those with power of attorney 56. The content from these various sources is consolidated by the digital healthcare system 12 and provided to the patient 28 using the interactive display device 26. The content may correspond to different electronic cognitive aids to improve orientation to the environment and provide a current location, for example, for the patient.

Particular embodiments of the subject matter have been described. Other embodiments are within the scope of the following claims.

What is claimed is:

1. A digital healthcare system for patient care, the digital healthcare system including at least hardware, software, data storage, and a network for interconnecting components of the system, including:
a cross-platform software for controlling an interactive display device for a patient being treated, the interactive display device facilitating access to a plurality of electronic cognitive aids, the interactive display device having a default home screen; and
a software application for receiving input from a plurality of different designated users via input devices to access the system and collaborate care for the patient, wherein the different designated users include at least one healthcare professional, caregiver, family member and/or power of attorney;
wherein the default home screen is customizable based on the collaborated care and/or user preferences using the software application.

2. The digital healthcare system of claim 1, wherein a screen of the interactive display includes a plurality of icons, wherein each icon navigates the patient to content provided exclusively by one of the different designated users.

3. The digital healthcare system of claim 1, wherein the interactive display displays a treatment plan provided by the healthcare professional.

4. The digital healthcare system of claim 1, wherein the patient is being treated for an acute condition or a chronic condition.

5. The digital healthcare system of claim 1, wherein the electronic cognitive aids include memory aids and communication aids.

6. The digital healthcare system of claim 1, wherein the electronic cognitive aids include audio and visual reminders.

7. The digital healthcare system of claim 1, wherein the electronic cognitive aids include at least one of speech generation, messaging, and artificial intelligence.

8. The digital healthcare system of claim 1, wherein the electronic cognitive aids include photos and/or videos.

9. The digital healthcare system of claim 1, wherein the electronic cognitive aids provide orientation including an identification of a current: location of the patient, a reason why the patient is at the current location, and an identification of a care team for the patient.

10. The digital healthcare system of claim 1, wherein the display customization includes identifying icons, determining a location or arrangement for each icon, and selecting a word or graphic symbol for each icon.

11. The digital healthcare system of claim 1, wherein the system is implemented using cloud-based computing.

12. The digital healthcare system of claim 1, wherein the system is a patient-facing system.

13. The digital healthcare system of claim 1, wherein the interactive display device is portable and is attachable to medical devices and equipment.

14. The digital healthcare system of claim 13, wherein the interactive display device is a bedside device.
